(19) United States
(12) Patent Application Publication
(10) Pub. No.: US 2024/0336875 A1
(43) Pub. Date: Oct. 10, 2024

(54) PACKAGED FABRIC REFRESHENING PRODUCT

(71) Applicant: The Procter & Gamble Company, Cincinnati, OH (US)

(72) Inventors: Crystal PHAM, Cincinnati, OH (US); Bing LENG, Singapore (SG); Yuri Volodymyrovych ROITER, Loveland, OH (US)

(21) Appl. No.: 18/624,419

(22) Filed: Apr. 2, 2024

Related U.S. Application Data
(60) Provisional application No. 63/457,783, filed on Apr. 6, 2023.

(51) Int. Cl. C11D 3/00; A61L 2/22; A61L 101/50; C11D 3/22; C11D 17/00

(52) U.S. Cl. CPC C11D 3/0068; A61L 2/22; C11D 3/222; C11D 17/0043; A61L 2101/50; A61L 2202/26

(57) ABSTRACT

A packaged fabric refreshening product for refreshening a fabric surface and reducing odor therefrom includes a liquid fabric freshening composition and a spray dispenser for dispensing the liquid fabric freshening composition. The liquid fabric freshening composition includes a liquid carrier and from 1 wt % to 5 wt % of cyclodextrin. The spray dispenser is a manually activated trigger-type spray dispenser, which, upon activation by a single movement of a trigger mechanism, is capable of continuously dispensing the liquid fabric freshening composition for a duration of at least 0.4 seconds. An effective amount of such liquid fabric freshening composition can be sprayed through such manually activated trigger-type spray dispenser onto a fabric surface, such as those on beddings, mattresses, sofas or carpets, which that is substantially planar with a minimal dimension of no less than 0.6 meters.

### TECHNICAL FIELD

The present disclosure relates to packaged fabric refreshening products for deodorizing and refreshening fabric-covered surfaces, and more particularly relatively large fabric-covered surfaces on household items such as beddings, mattresses, sofas, and carpets.

### BACKGROUND

Household items such as beddings, mattresses, sofas and carpets have large fabric-covered surfaces, which tend to accumulate dirt/sweat/sebum and emit malodors over time. It is a particular hassle to wash or clean the fabric covers on these household items, due to the difficulty in taking such fabric covers off and putting them back on. Therefore, packaged fabric refreshening products with aerosol or trigger sprayers have been made available for consumers to directly spray liquid fabric refreshening compositions onto the large fabric-covered surfaces of these household items, so as to restore their freshness and reduce malodor, without having to take the fabric covers off for washing or dry cleaning.

However, fabric refreshening products with conventional aerosol or trigger sprayers are difficult to use and do not offer speedy and efficient treatment of beddings, mattresses, sofas and carpets with such large fabric-covered surfaces. For example, a consumer needs to spray 20 or more times in order to treat the entire surface of a bedding with the fabric refreshening product, which is both slow and exhausting.

Further, conventional sprayers often dispense intermittent bursts of fabric refreshening products, which cannot uniformly or evenly cover the fabric surface, leaving some spots that are dry and untreated, while other spots are soaking wet. Consequently, the treated fabric surface cannot be used immediately afterwards because the wet spots will take a while to dry. The slowness in drying and the unpleasant feel associated with wet spots are key tension points that have discouraged or deterred consumers from using such fabric refreshening products as often as they would like. More importantly, the uneven distribution of a fabric refreshening product on the treated fabric surface may limit its overall deodorizing and refreshening effect, e.g., the untreated dry spots can continue to emit malodors.

There is therefore a need for an improved fabric refreshening product that is easy to use, which provides a more efficient way for deodorizing and refreshing large fabric surfaces, such as those typically seen on beddings, mattresses, sofas, and carpets.

There is also a need for an improved fabric refreshening product that provides effective and uniform coverage of the large fabric surfaces on beddings, mattresses, sofas, and carpets. Specifically, it is desirable that such improved fabric refreshening product enables fast drying of the fabric surfaces after treatment and allows immediate usage of such household items, which in turn encourages and incentivizes consumers to use such product more frequently.

There is further a need for an improved fabric refreshening product that offers more effective and efficient deodorization and refreshening of fabric-covered surfaces.

### SUMMARY

The present disclosure relates to a packaged fabric refreshening product comprising:
a) a liquid fabric freshening composition; and
b) a spray dispenser for dispensing the liquid fabric freshening composition,

wherein the liquid fabric freshening composition comprises a liquid carrier and from 1 wt % to 5 wt % of cyclodextrin; and wherein the spray dispenser is a manually activated trigger-type spray dispenser, which, upon activation by a single movement of a trigger mechanism, is capable of continuously dispensing the liquid fabric freshening composition for a duration of at least 0.4 seconds.

Specifically, the liquid fabric freshening composition may comprise from 1.2 wt % to 3 wt %, or from 1.5 wt % to 2.5 wt %, of cyclodextrin. Optionally, the cyclodextrin can be selected from the group consisting of alpha-cyclodextrin, beta-cyclodextrin, gamma-cyclodextrin, and derivatives and/or mixtures thereof; wherein optionally the cyclodextrin is selected from the group consisting of hydroxy-propyl alpha-cyclodextrin (HPACD), methylated alpha-cyclodextrin (MACD), methylated beta-cyclodextrin (MBCD), hydroxy-ethyl beta-cyclodextrin (MEBCD), hydroxy-propyl beta-cyclodextrin (HPBCD), and derivatives and/or mixtures thereof. The above-mentioned liquid fabric freshening composition may further comprise one or more ingredients selected from the group consisting of: of: perfumes; cyclodextrin-compatible surfactants; cyclodextrin-compatible antimicrobial actives; and any mixtures thereof.

Specifically, the above-mentioned spray dispenser is capable of continuously dispensing the liquid fabric freshening composition for a duration of from 0.4 seconds to 2 seconds, or from 0.5 seconds to 1.8 seconds, or from 0.6 seconds to 1.5 second upon activation by a single movement of the trigger mechanism. Optionally, such spray dispenser comprises: (1) a container body in which the liquid fabric freshening composition is contained; (2) a nozzle; and (3) an ejector in fluid communication with such container body and such nozzle for suctioning the liquid fabric freshening composition from the container body and for ejecting the liquid fabric freshening composition forward through a nozzle upon activation by the trigger mechanism.

Specifically, the liquid fabric freshening composition dispensed by the above-mentioned manually activated trigger-type spray dispenser is characterized by an average droplet size (DV50) of from 50 microns to 120 microns, optionally from 60 microns to 110 microns, optionally from 70 microns to 100 microns.

The present disclosure also relates to a method of refreshening a fabric surface and reducing odor therefrom, comprising the steps of:
(a) providing a liquid fabric freshening composition that comprises a liquid carrier and from 1 wt % to 5 wt % of cyclodextrin; and (
b) spraying an effective amount of such liquid fabric freshening composition onto a fabric surface by using a manually activated trigger-type spray dispenser,

wherein such fabric surface is substantially planar with a minimal dimension of no less than 0.6 meters; and wherein such manually activated trigger-type spray dispenser, upon activation by a single movement of a trigger mechanism, is capable of continuously dispensing the liquid fabric freshening composition for a duration of at least 0.4 seconds.

Specifically, the above-mentioned fabric surface is selected from the group consisting of beddings, mattresses, sofas, and carpets.

### BRIEF DESCRIPTION OF THE DRAWING

FIG. 1 is a front view of a manually activated trigger-type spray dispenser, according to an example of the present disclosure.

### DETAILED DESCRIPTION

The packaged fabric refreshening product of the present disclosure employs a manually activated trigger-type spray dispenser with a unique trigger mechanism that allows for continuous and sustained release of a liquid fabric freshening composition for a duration of at least 0.4 seconds, optionally from 0.4 seconds to 2 seconds, or from 0.5 seconds to 1.8 seconds, or from 0.6 seconds to 1.5 second upon a single activation of the trigger mechanism. This spray dispenser is easy to use and provides a faster and more efficient way for treating large fabric surfaces, such as those typically seen on beddings, mattresses, sofas, and carpets. This spray dispenser also enables fast drying of the treated fabric surfaces by dispensing the liquid fabric freshening composition at smaller droplet sizes to form a finer mist for effectively and uniformly covering the fabric surfaces to be treated.

More importantly, the present disclosure combines such spray dispenser with a specifically designed liquid fabric freshening composition, which contains a higher amount of an odor-absorbing compound, i.e., from 1 wt % to 5 wt % (or from 1.2 wt % to 3 wt %, or from 1.5 wt % to 2.5 wt %) of cyclodextrin. Surprisingly and unexpectedly, such a combination results in synergistic improvement of the odor control and malodor reduction performance of the resulting packaged fabric refreshening product.

### Manually Activated Trigger-Type Spray Dispenser

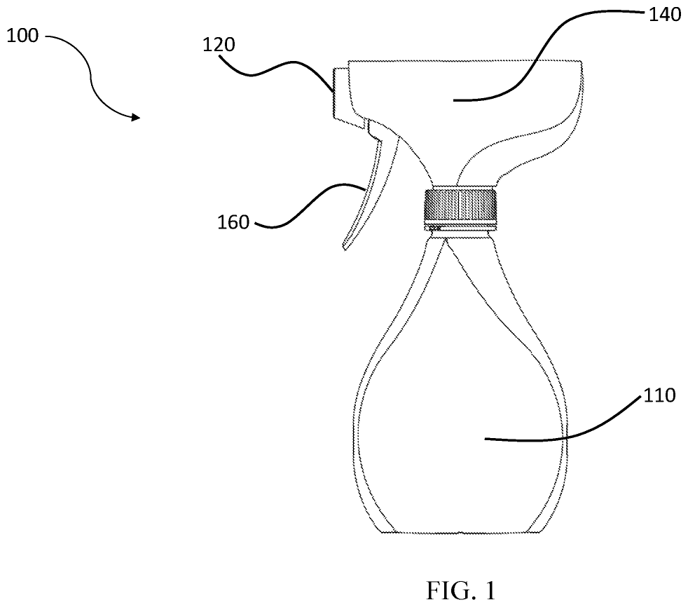

The spray dispenser employed by the packaged fabric refreshening product of the present disclosure is a manually activated trigger-type spray dispenser capable of continuous discharging the liquid fabric freshening composition. The term “continuous” as used herein refers to discharge of liquid by a trigger-activated spray dispenser not only at the time when the trigger is pulled, but also at a time afterwards (i.e., when the trigger pulling has stopped). FIG. 1 is a front view of a manually activated trigger-type spray dispenser 100, according to a specific example of the present disclosure, which comprises: (1) a container body 110 in which a liquid fabric freshening composition (not seen) is contained; (2) a nozzle 120 provided with an ejecting hole (not seen) configured to inject the liquid fabric freshening composition forward; and (3) an ejector 140 in fluid communication with the container body 110 and the nozzle 120 for suctioning the liquid fabric freshening composition from the container body 110 and for ejecting the liquid fabric freshening composition forward through the nozzle 120 upon activation by a trigger mechanism 160 that is configured to be movable rearward while being biased forward.

The above-mentioned trigger-activated ejector is capable of continuously ejecting a relatively large amount of the liquid fabric refreshening composition, such as those disclosed in US Patent Application Publication No. 2017/0216863A1, US Patent Application Publication No. 2018/0369842A1, Japanese Patent No. 6971190B, and US Patent Application Publication No. 2022/0401982A1. For example, the trigger-activated ejector may comprise: an ejector main body attached to the above-mentioned container body; a vertical supply pipe extending in an up-and-down direction and used to suck up the liquid fabric freshening composition contained in the container body; an ejection barrel extending forward from the vertical supply pipe, while an internal area of the ejection barrel communicating with an internal area of the vertical supply pipe. The above-mentioned trigger mechanism includes a trigger that extends downward from the ejection barrel and arranged so as to be movable rearward in a state where the trigger receives forward force. The trigger mechanism is configured to lead the liquid fabric freshening composition from the internal area of the vertical supply pipe into the ejection barrel in accordance with rearward movement of the trigger and to eject the liquid fabric freshening composition from the internal area of the ejection barrel toward the ejection hole. In addition, the nozzle is provided with: a cylinder extending in a front-and-rear direction, while an internal area of the cylinder is communicating with the internal area of the ejection barrel through a supply hole; a plunger disposed in the cylinder so as to be movable rearward in a state where the plunger receives forward force; and a communication hole allowing the internal area of the cylinder and the ejection hole to communicate with each other.

When the trigger is pulled rearward, the liquid fabric freshening composition is sucked up through the vertical supply pipe from the internal area of the container body and is then led into the ejection barrel, which subsequently ejects the liquid fabric freshening composition into the cylinder through the supply hole. Accordingly, the plunger inside the cylinder can be moved rearward while countering the forward force. In addition, at this time, the liquid fabric freshening composition can be supplied from the internal area of the ejection barrel through the communication hole to the ejection hole and can be dis-charged outward from the ejection hole.

In this way, every time the operation of pulling the trigger is performed, the liquid fabric freshening composition is discharged from the ejection hole; the plunger is moved rearward; and thus the liquid fabric freshening composition can be stored or filled in the cylinder. At the time the operation of pulling the trigger is stopped, the supply of the liquid fabric freshening composition into the ejection barrel is stopped, while the plunger starts moving forward through the forward force. Accordingly, the liquid fabric freshening composition stored or filled in the cylinder can be continuously and uninterruptedly discharged from the ejection hole through the communication hole. Thus, the liquid fabric freshening composition is discharged not only at the time the operation of rearward pulling the trigger is performed but also at a time the operation of the trigger is not performed (i.e., after the trigger pulling has stopped), resulting in a continuous discharge of the liquid fabric freshening composition.

Further, although the plunger is configured to move forward up to the most-forward position thereof if the trigger is not pulled again during the forward movement of the plunger, it is also possible to repeat the operation of pulling the trigger before the plunger reaches the most-forward position. In this case, while the plunger repeats forward and rearward movements due to the repeated pulling of the trigger, each of the forward and rearward movements of the plunger allows it to travel rearward little by little, at an approximately constant distance overall. As a result, the liquid fabric freshening composition can gradually be stored or filled in the cylinder, which can then be discharged from the ejection hole to form a continuous spray when the operation of pulling the trigger is stopped but the plunger continues to move forward through the forward force.

Specifically, the spray dispenser of the present disclosure allows for continuous and sustained delivery of the liquid fabric freshening composition for a duration of at least 0.4 seconds, optionally from 0.4 seconds to 2 seconds, or from 0.5 seconds to 1.8 seconds, or from 0.6 seconds to 1.5 second, upon a single activation of the trigger. By employing such a continuous spray dispenser, the present disclosure provides packaged fabric refreshening products that are easy to use and enable a more efficient way for deodorizing and refreshing large fabric surfaces, such as those typically seen on ‘beddings, mattresses, sofas, and carpets. For example, for beddings having a substantially planar fabric surface with a minimal dimension of no less than 0.6 meter, optionally no less than 0.8 meters, optionally no less than 1 meter, optionally no less than 1.3 meters, which may require a consumer to spray 20 or more times in order to treat its entire surface when using a conventional fabric refreshening product that employs a non-continuous trigger-activated spray dispenser, the consumer only needs to spray about 8 times to cover the same fabric surface when using the packaged fabric refreshening product of the present disclosure with the continuous trigger-activated spray dispenser.

The spray dispenser of the present disclosure may be further characterized by an output per stroke of from about 0.5 ml to about 2 ml, or from about 0.8 ml to about 1.5 ml, or from about 1 ml to about 1.2 ml, when operating at a speed of 90 strokes per minute (SPM).

Preferably, such spray dispenser dispenses the liquid fabric freshening composition in form of a fine mist characterized by an average droplet size (DV50) ranging from about 50 microns to about 120 microns, optionally from about 60 microns to about 110 microns, optionally from about 70 microns to about 100 microns. Optionally, the fine mist formed by the dispensed fabric freshening composition is also characterized by: (a) DV90 ranging from about 80 microns to about 200 microns, or from about 90 microns to about 180 microns, or from about 100 microns to about 150 microns; and/or (b) DV10 ranging from 30 microns to 80 microns, or from 35 microns to 70 microns, or from 40 microns to 60 microns. Further, such a fine mist may define: (1) a spray pattern with a diameter ranging from 14 cm to about 20 cm, optionally from about 15 cm to about 18 cm, optionally from about 16 cm to about 17 cm; and/or (2) a cone angle ranging from 50 degree to 65 degree, optionally from 55 degree to 60 degree. Such a fine mist formed by the dispensed fabric freshening composition provides effective and uniform coverage of the large fabric surfaces on beddings, mattresses, sofas, and carpets. It also enables fast drying of the fabric surfaces after treatment and allows immediate usage of such household items.

### Liquid Fabric Freshening Composition

The liquid fabric refreshening composition used in the present disclosure comprise cyclodextrin, which is an odor-absorbing active, at a level ranging from 1 wt % to 5 wt %, or from 1.2 wt % to 3 wt %, or from 1.5 wt % to 2.5 wt %, by total weight of such liquid fabric refreshening composition. Without being bound by any theory, it is believed that a liquid fabric refreshening composition with such a relatively higher level of cyclodextrin, when used in combination with the continuous trigger-activated spray dispenser of the present disclosure, surprisingly and unexpectedly delivers a synergistically improved odor control and malodor reduction benefit (in comparison with similar packaged products either with a lower level of cyclodextrin or with a non-continuous trigger-activated spray dispenser).

The liquid fabric refreshening composition of the present disclosure also comprises a liquid carrier, in which the cyclodextrin is dissolved. Optionally, it further comprises one or more ingredients selected from the group consisting of: perfumes; cyclodextrin-compatible surfactants; cyclodextrin-compatible antimicrobial actives; and any mixtures thereof, as disclosed hereinafter.

### (A) Cyclodextrin

As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in donut-shaped rings. The specific coupling and conformation of the glucose units give the cyclodextrins a rigid, conical molecular structure with hollow interiors of specific volumes. The “lining” of each internal cavity is formed by hydrogen atoms and glycosidic bridging oxygen atoms; therefore, this surface is more hydrophobic. The unique shape and physical-chemical properties of the cavity enable the cyclodextrin molecules to absorb (form inclusion complexes with) organic molecules or parts of organic molecules which can fit into the cavity. Many odorous molecules can fit into the cavity including many malodorous molecules and perfume molecules. Therefore, cyclodextrins, and especially mixtures of cyclodextrins with different size cavities, can be used to control odors caused by a broad spectrum of organic odoriferous materials, which may, or may not, contain reactive functional groups. The complexation between cyclodextrin and odorous molecules occurs rapidly in the presence of water. However, the extent of the complex formation also depends on the polarity of the absorbed molecules. In an aqueous solution, strongly hydrophilic molecules (those which are highly water-soluble) are only partially absorbed, if at all. Therefore, cyclodextrin does not complex effectively with some very low molecular weight organic amines and acids when they are present at low levels on wet fabrics. As the water is being removed however, e.g., the fabric is being dried off, some low molecular weight organic amines and acids have more affinity and will complex with the cyclodextrins more readily.

The cavities within the cyclodextrin in the solution of the present disclosure should remain essentially unfilled (i.e., the cyclodextrin remains uncomplexed) while in solution, in order to allow the cyclodextrin to absorb various odor molecules when the solution is applied to a surface. Nonderivatised (native) beta-cyclodextrin can be present at a level up to its solubility limit of about 1.85% (about 1.85 g in 100 grams of water) at room temperature. Nonderivatised beta-cyclodextrin is generally not preferred when the composition contains surfactant since it affects the surface activity of most of the preferred surfactants that are compatible with the derivatized cyclodextrins.

The cyclodextrins used in the present disclosure may be highly water-soluble such as, alpha-cyclodextrin and/or derivatives thereof, gamma-cyclodextrin and/or derivatives thereof, derivatised beta-cyclodextrins, and/or mixtures thereof. The derivatives of cyclodextrin consist mainly of molecules wherein some of the OH groups are converted to OR groups. Cyclodextrin derivatives include, e.g., those with short chain alkyl groups such as methylated cyclodextrins, and ethylated cyclodextrins, wherein R is a methyl or an ethyl group; those with hydroxyalkyl substituted groups, such as hydroxypropyl cyclodextrins and/or hydroxyethyl cyclodextrins, wherein R is a —CH2—CH (OH)—CH3, CH(CH3)—CH2—OH, or a —CH2CH2—OH group; branched cyclodextrins such as maltose-bonded cyclodextrins; cationic cyclodextrins such as those containing 2-hydroxy-3-(dimethylamino) propyl ether, wherein R is CH2—CH (OH)—CH2—N(CH3)2 which is cationic at low pH; quaternary ammonium, e.g., 2-hydroxy-3 (trimethylammonio) propyl ether chloride groups, whereinR is CH2—CH(OH)—CH2—N+(CH3)3 Cl—; anionic cyclodextrins such as carboxymethyl cyclodextrins, cyclodextrin sulfates, and cyclodextrin succinylates; amphoteric cyclodextrins such as carboxymethyl/quaternary ammonium cyclodextrins; cyclodextrins wherein at least one glucopyranose unit has a 3-6-anhydro-cyclomalto structure, e.g., the mono-3-6-anhydrocyclodextrins, and mixtures thereof.

Highly water-soluble cyclodextrins are those having water solubility of at least about 10 g in 100 ml of water at 25° C., preferably at least about 20 g in 100 ml of water, more preferably at least about 25 g in 100 ml of water at room temperature. The availability of solubilized, uncomplexed cyclodextrins is essential for effective and efficient odor control performance. Solubilized, water-soluble cyclodextrin can exhibit more efficient odor control performance than non-water-soluble cyclodextrin when deposited onto surfaces, especially fabric.

Examples of preferred water-soluble cyclodextrin derivatives suitable for use herein are hydroxypropyl alpha-cyclodextrin (HPACD), methylated alpha-cyclodextrin (MACD), methylated beta-cyclodextrin (MBCD), hydroxyethyl beta-cyclodextrin (HEBCD), and hydroxypropyl beta-cyclodextrin (HPBCD). Hydroxyalkyl cyclodextrin derivatives preferably have a degree of substitution of from about 1 to about 14, more preferably from about 1.5 to about 7, wherein the total number of OR groups per cyclodextrin is defined as the degree of substitution. Methylated cyclodextrin derivatives typically have a degree of substitution of from about 1 to about 18, preferably from about 3 to about 16. A known methylated beta-cyclodextrin is heptakis-2,6-di-O-methyl-β-cyclodextrin, commonly known as DIMEB, in which each glucose unit has about 2 methyl groups with a degree of substitution of about 14. A preferred, more commercially available, methylated beta-cyclodextrin is a randomly methylated beta-cyclodextrin, commonly known as RAMEB, having different degrees of substitution, normally of about 12.6. RAMEB is more preferred than DIMEB, since DIMEB affects the surface activity of the preferred surfactants more than RAMEB. The preferred cyclodextrins are available, e.g., from Cerestar USA, Inc. and Wacker Chemicals (USA), Inc.

It may be preferable to use a mixture of cyclodextrins. The mixture may include alkoxylated and/or nonalkoxylated cyclodextrins. Such mixtures absorb odors more broadly by complexing with a wider range of odoriferous molecules having a wider range of molecular sizes. Preferably at least a portion of the cyclodextrins is alpha-cyclodextrin and its derivatives thereof, gamma-cyclodextrin and its derivatives thereof, and/or derivatised beta-cyclodextrin, more preferably a mixture of alpha-cyclodextrin, or an alpha-cyclodextrin derivative, and derivatised beta-cyclodextrin, even more preferably a mixture of derivatized alpha-cyclodextrin and derivatised beta-cyclodextrin, most preferably a mixture of hydroxypropyl alpha-cyclodextrin and hydroxypropyl beta-cyclodextrin, and/or a mixture of methylated alpha-cyclodextrin and methylated beta-cyclodextrin.

### (B) Liquid Carrier

The liquid fabric refreshening composition of the present disclosure includes a liquid carrier, in which the above-mentioned cyclodextrin is dissolved. The liquid carrier may be water, which may be distilled, deionized, tap, or further purified forms of water. Water may be present in any sufficient amount for the composition to be an aqueous solution. Specifically, water may be present in an amount from about 80 wt % to 99.5 wt %, alternatively from about 85 wt % to about 99.5 wt %, alternatively from about 92 wt % to about 99.5 wt %, alternatively from about 95 wt %, by total weight of the liquid fabric refreshening composition.

Low molecular weight monohydric alcohols (e.g., ethanol, methanol, and isopropanol, or polyols, such as ethylene glycol and propylene glycol) can also be useful as the liquid carrier, either alone or in combination with water. In some instances, the level of monohydric alcohol may be less than about 20 wt. %, or less than about 15 wt %, or less than about 10 wt %, or less than about 6 wt %, alternatively less than about 3 wt %, alternatively less than about 1 wt %, by weight of the liquid fabric refreshening composition.

### (C) Other Ingredients

The liquid fabric refreshening composition of the present disclosure may optionally provide a “scent signal” in the form of a pleasant odor which signals the removal of malodor from fabrics. The scent signal is designed to provide a fleeting perfume scent and is not designed to be overwhelming or to be used as an odor masking ingredient. When perfume is added as a scent signal, it is added only at very low levels, e.g., from about 0 wt % to about 0.5 wt %, preferably from about 0.003 wt % to about 0.3 wt %, more preferably from about 0.005 wt % to about 0.2 wt %, by total weight of the liquid fabric refreshening composition.

Perfume can also be added as a more intense odor in product and on surfaces. When stronger levels of perfume are preferred, relatively higher levels of perfume (for example, from 0.1 wt % to 2 wt %, or from 0.2 wt % to 1.5 wt %, or from 0.3 wt % to 1 wt %) can be added. It is essential, however, that the perfume be added at a level wherein even if all of the perfume in the composition were to complex with the cyclodextrin molecules, there will still be an effective level of uncomplexed cyclodextrin molecules present in the solution to provide adequate odor control. In order to reserve an effective amount of cyclodextrin molecules for odor control, perfume is typically present at a level wherein less than about 90% of the cyclodextrin complexes with the perfume, preferably less than about 50% of the cyclodextrin complexes with the perfume, more preferably, less than about 30% of the cyclodextrin complexes with the perfume, and most preferably, less than about 10% of the cyclodextrin complexes with the perfume. The cyclodextrin to perfume weight ratio should be greater than about 8:1, preferably greater than about 10:1, more preferably greater than about 20:1, even more preferably greater than 40:1 and most preferably greater than about 70:1.

Any type of perfume raw materials (PRMs) can be incorporated into the liquid fabric refreshening composition of the present disclosure. Various PRMs may be used. The perfume mixture may comprise one or more of the following perfume raw materials: fragrant essential oils; natural and synthetic aroma compounds; pro-perfumes; materials supplied with the fragrant essential oils, aroma compounds, and/or pro-perfumes, including stabilizers, diluents, processing agents, and contaminants; and any material that commonly accompanies fragrant essential oils, aroma compounds, and/or pro-perfumes.

Preferably the perfume is hydrophilic and is composed predominantly of ingredients selected from two groups of ingredients, namely, (a) hydrophilic perfume ingredients having a ClogP of less than about 3.5, more preferably less than about 3.0, and (b) perfume ingredients having significant low detection threshold, and mixtures thereof. Typically, at least about 50%, preferably at least about 60 wt %, more preferably at least about 70 wt %, and most preferably at least about 80 wt % of the perfume is composed of perfume ingredients of the above groups (a) and (b).

Non-limiting examples of hydrophilic perfume ingredients of the above group (a) are allyl amyl glycolate, allyl caproate, amyl acetate, amyl propionate, anisic aldehyde, anisyl acetate, anisole, benzaldehyde, benzyl acetate, benzyl acetone, benzyl alcohol, benzyl formate, benzyl iso valerate, benzyl propionate, beta gamma hexenol, calone, camphor gum, laevo-carveol, d-carvone, laevo-carvone, cinnamic alcohol, cinnamyl acetate, cinnamic alcohol, cinnamyl formate, cinnamyl propionate, cis-jasmone, cis-3-hexenyl acetate, coumarin, cuminic alcohol, cuminic aldehyde, Cyclal C, cyclogalbanate, dihydrocuginol, dihydro isojasmonate, dimethyl benzyl carbinol, dimethyl benzyl carbinyl acetate, ethyl acetate, ethyl aceto acetate, ethyl amyl ketone, cthyl anthranilate, ethyl benzoate, ethyl butyrate, ethyl cinnamate, ethyl hexyl ketone, ethyl maltol, ethyl-2-methyl butyrate, ethyl methylphenyl glycidate, ethyl phenyl acetate, ethyl salicylate, ethyl vanillin, eucalyptol, eugenol, eugenyl acetate, eugenyl formate, eugenyl methyl ether, fenchyl alcohol, flor acetate (tricycle decenyl acetate), fructone, frutene (tricycle decenyl propionate), geraniol, geranyl oxyacetaldehyde, heliotropin, hexenol, hexenyl acetate, hexyl acetate, hexyl formate, hinokitiol, hydratropic alcohol, hydroxycitronellal, hydroxycitronellal diethyl acetal, hydroxycitronellol, indole, isoamyl alcohol, iso cyclo citral, isoeugenol, isocugenyl acetate, isomenthone, isopulegyl acetate, isoquinoline, keone, ligustral, linalool, linalool oxide, linalyl formate, lyral, menthone, methyl acetophenone, methyl amyl ketone, methyl anthranilate, methyl benzoate, methyl benzyl acetate, methyl cinnamate, methyl dihydrojasmonate, methyl cugenol, methyl heptenone, methyl heptine carbonate, methyl heptyl ketone, methyl hexyl ketonc, methyl isobutenyl tetrahydropyran, methyl-N-methyl anthranilate, methyl beta naphthyl ketone, methyl phenyl carbinyl acetate, methyl salicylate, nerol, nonalactone, octalactone, octyl alcohol (octanol-2), para-anisic aldehyde, para-cresol, para-cresyl methyl ether, para hydroxy phenyl butanone, para-methoxy acctophenone, para-methyl acetophenone, phenoxy ethanol, phenoxyethyl propionate, phenyl acetaldehyde, phenylacetaldehyde diethyl ether, phenylethyl oxyacetaldehyde, phenyl ethyl acetate, phenyl ethyl alcohol, phenyl ethyl dimethyl carbinol, prenyl acetate, propyl butyrate, pulegone, rose oxide, safrole, terpineol, vanillin, viridine, and mixtures thereof.

Non-limiting examples of perfume ingredients that do not belong to group (a) above, but have significant low detection threshold and are therefore useful in the liquid fabric refreshening composition of the present disclosure, are selected from the group consisting of ambrox, bacdanol, benzyl salicylate, butyl anthranilate, cetalox, damascenone, alpha-damascone, gamma-dodecalactone, cbanol, herbavert, cis-3-hexenyl salicylate, alpha-ionone, beta-ionone, alphaisomethylionone, lilial, methyl nonyl ketone, gamma-undecalactone, undecylenic aldehyde, and mixtures thereof. These materials are preferably present at low levels in addition to the hydrophilic ingredients of group (a), typically less than about 20%, preferably less than about 15 wt %, more preferably less than about 10 wt %, by total weight of the perfume ingredients in the composition.

There are also hydrophilic perfume ingredients of group (a) that have a significantly low detection threshold, which are especially useful in the liquid fabric refreshening composition of the present disclosure. Examples of these perfume ingredients are allyl amyl glycolate, ancthole, benzyl acetone, calone, cinnamic alcohol, coumarin, cyclogalbanate, Cyclal C, cymal, 4-decenal, dihydro isojasmonate, ethyl anthranilate, ethyl-2-methyl butyrate, ethyl methylphenyl glycidate, ethyl vanillin, eugenol, flor acetate, florhydral, fructone, frutene, heliotropin, keone, indole, iso cycle citral, isocugenol, lyral, methyl heptane carbonate, linalool, methyl anthranilate, methyl dihydrojasmonate, methyl isobutenyl tetrahydropyran, methyl beta naphthyl ketone, beta naphthol methyl ether, nerol, para-anisic aldehyde, para hydroxy phenyl butanone, phenyl acetaldehyde, vanillin, and mixtures thereof.

The liquid fabric refreshening composition of the present disclosure may optionally comprise one or more cyclodextrin-compatible surfactants, which may provide various additional benefits such as permitting the liquid fabric refreshening composition to spread readily and more uniformly on hydrophobic surfaces like polyester and nylon, allowing it to dry faster, enabling penetration into hydrophobic, oily soil better for improved malodor control, providing improved “in-wear” electrostatic control, and the like. Such cyclodextrin-compatible surfactants may be anionic surfactants, nonionic surfactants, cationic surfactants, amphoteric surfactants, zwitterionic surfactants, and mixtures thereof. Non-limiting examples of cyclodextrin-compatible nonionic surfactants include block copolymers of ethylene oxide and propylene oxide. Suitable block polyoxyethylene-polyoxypropylene polymeric surfactants, that are compatible with most cyclodextrins, include those based on ethylene glycol, propylene glycol, glycerol, trimethylolpropane and ethylenediamine as the initial reactive hydrogen compound. Certain of the block polymer surfactant compounds are commercially available from BASF under the tradenames Pluronic® and Tetronic®. Other examples of cyclodextrin-compatible surfactants include: (1) the Silwet® surfactants, which are commercially available from OSi Specialties, Inc. (Danbury, Connecticut), such as Silwet® L-7600, L-7602, L-7604, L-7605, L-7622, L-7657, and mixtures thereof; (2) the Dowfax® surfactants available from the Dow Chemical Company, such as Dowfax 3B2; (3) surfactant derivatives of hydrogenated castor oil (HCO), such as ethoxylated hydrogenated castor oil commercially available as BASOPHOR™ from BASF and CREMOPHOR™ from Sigma Aldrich. Typical levels of cyclodextrin-compatible surfactants are from about 0.01 wt % to about 5 wt %, or from about 0.03 wt % to about 4 wt %, or from about 0.05 wt % to about 3 wt %, by total weight of the liquid fabric refreshening composition of the present disclosure.

The liquid fabric refreshening composition of the present disclosure may optionally comprise one or more cyclodextrin-compatible antimicrobial actives, which provide protection against organisms that become attached to the treated fabric surfaces but without forming complexes with the cyclodextrin in the liquid fabric refreshening composition. Suitable antimicrobial actives may include antibacterial halogenated compounds, quaternary compounds, and phenolic compounds. Examples of suitable halogenated compounds are biguanides and bis biguanide compounds, such as 1, 1′-hexamethylene bis (5-(p-chlorophenyl) biguanide), which is commonly known as chlorhexidine, and its salts (e.g., with hydrochloric, acetic and gluconic acids); poly (hexamethylene biguanide) hydrochloride; bis-biguanide alkanes. Examples of suitable quaternary compounds are di (C8-C12) dialkyl dimethyl ammonium chloride, such as didecyldimethylammonium chloride (Bardac 22 or DDAC), and dioctyldimethylammonium chloride (Bardac 2050). Typical concentrations for these antimicrobial actives range from about 0.001 wt % to about 1 wt %, or from about 0.05 wt % to about 0.5 wt %, or from about 0.1 wt % to about 0.3 wt % (or below any upper limit set by the laws and regulations of the relevant markets), by total weight of the liquid fabric refreshening composition.

The liquid fabric refreshening composition of the present disclosure may include a buffer system for maintaining a desired pH. The liquid fabric refreshening composition of the present disclosure may have a pH from about 4 to about 9, alternatively from about 4 to about 8.5, alternatively from about 4 to about 6.9, alternatively about 4 to about 6.7. The buffer system may comprise one or more buffering agents, specifically acidic buffering agents, such as dibasic acids, carboxylic acids, dicarboxylic acids, tricarboxylic acids, polycarboxylic acids. Preferably, the buffering agents are selected from the group consisting of: citric acid, maleic acid, polyacrylic acid, and combinations thereof. It has been found that buffer systems that include a buffering agent selected from the group consisting of: citric acid, maleic acid, polyacrylic acid, and combinations thereof provide stable freshening compositions with prolonged shelf life. The liquid fabric refreshening composition may contain at least about 0.001 wt %, alternatively at least about 0.01 wt %, of such a buffering agent by total weight of the composition. The composition may also contain no more than about 2 wt %, alternatively no more than about 0.75 wt %, alternatively no more than about 0.5 wt %, of such a buffering agent, by total weight of the composition.

Adjuvants can be optionally added to the liquid fabric refreshening composition herein for their known purposes. Such adjuvants include, but are not limited to, water soluble metallic salts, antistatic agents, insect and moth repelling agents, colorants, antioxidants, and mixtures thereof.

In addition, the liquid fabric refreshening composition of the present disclosure may include preservatives; antimicrobial compounds; materials that acts to condition, modify, or otherwise modify the environment (e.g., to assist with sleep, wake, respiratory health, and like conditions); deodorants or malodor control compositions (e.g., odor neutralizing materials such as reactive aldehydes, odor blocking materials, odor masking materials, or sensory modifying materials such as ionones), and other malodor removal compounds such as polyamines, including polyethylene imines.

### TEST METHODS

### Test 1: Measurement of the Spray Duration

The spray duration of a sample trigger-type spray dispenser is determined using a Malvern Spraytec particle size analyzer and Spraytec software. Both are available from Malvern Instruments, Ltd, UK.

A 750 mm lens is used with a minimum and maximum particle size detection of 2 and 2000 microns, respectively. Nozzle of the sample spray dispenser is positioned 140 mm from the laser beam, using a 100 mm path length. A particulate refractive index of 1.33 and dispersant refractive index of 1.00 are selected. A residual of 0.42 is selected, with the extinction analysis set to Off and multiple scatter set to On. The Scatter start is set to 1, scatter end is set to 33, and scattering threshold is set to 1. The testing temperature is about 22.5 degree Celsius.

Duration of spray is recorded in the measurement in the form of ‘span’, referring to the first moment of detection of particles passing through the laser to the last particle throughout one spray. Each spray is done by automated actuation at the timing of 90 sprays per minute.

### Test 2: Liquid Droplet Size Measurement

The particle size or diameter of liquid droplets dispensed by a sample trigger-type spray dispenser is determined using a Malvern Spraytec particle size analyzer and Spraytec software. Both are available from Malvern Instruments, Ltd, UK.

A 750 mm lens is used, having minimum and maximum particle size detections of 2 and 2000 microns, respectively. Nozzle of the sample spray dispenser is positioned 140 mm from the laser beam, using a 100 mm path length. A particulate refractive index of 1.33 and dispersant refractive index of 1.00 are selected. A residual of 0.42 is selected, with the extinction analysis set to Off and multiple scatter set to On. The Scatter start is set to 1, scatter end is set to 33, and scattering threshold is set to 1. The testing temperature is about 22.5 degree Celsius.

One of skill will consider the DV (50) measurement, meaning that 50 percent of the droplets have a mean particle diameter less than the value indicated. Likewise, one of skill will consider the DV (90) measurement, meaning that 90 percent of the droplets have a mean particle diameter less than the value indicated.

### Test 3: Paper Trap Method for Determining Non-Volatiles Delivered by Packaged Fabric Refreshening Products

The paper trap method is performed by directing the center of a spray cone coming from a sample packaged fabric refreshing product with a spray dispenser through a round hole in plastic sheet and catching the central part of the spray passing through the round hole by a paper trap positioned behind the round hole. This method measures how focused the delivery of non-volatiles (as dissolved in a sprayed material but left after sufficient drying, which may include some unevaporated perfumes) is in the center of a spray pattern produced by the sample packaged fabric refreshing product with the spray dispenser, which is indicative of effective odor control and malodor reduction by such sample packaged fabric refreshing product. Specifically, the more non-volatiles are delivered by the sample packaged fabric refreshening product in the central part of the spray pattern through the round hole, the more focused (and therefore more effective and efficient) its odor control and malodor reduction performance.

Specifically, a sample packaged fabric refreshing product equipped with a specific trigger-type spray dispenser and filled with a specific liquid fabric freshening composition between 75% and 90% of its capacity is tilted at 45 degrees (Parameter A) and mounted in a vice preventing it from accidental shift of position. The nozzle of the spray dispenser is positioned in 7 7/8″ (20 cm, Parameter B) from a transparent 12″ (width, horizontal side)×24″ (height)×¼″ (thickness) plastic sheet (e.g. polymethylmethacrylate sheet) tilted at 45 degrees (Parameter C) in the direction opposite from the tilt of the spray dispenser, forming a 90 degree angle (Parameter D) between the spraying direction and the plastic sheet, with a 1″ diameter round hole in the center of the transparent sheet. Plastic sheet is held robustly in place by, for example, a laboratory stand with clamps. Before attaching the paper trap, position of the spray dispenser relative to the plastic sheet is adjusted by tentative spraying and correcting its angle and position, so that the center of the spray cone would come through the hole. After such adjustments, parameters A-D should not deviate more than 5% from the above-stated original values.

A paper trap made of a liquid-adsorbing paper 11″ (width)×6″ (height) in size, such as

“The Quicker Picker Upper” Select-a-Size Bounty (P&G) for liquids, is used in this test. Paper trap is hung by upper corners along the paper width (longer side) onto a transparent plastic sheet with a pair of extra-large binder clips from the opposite from sprayer side behind the hole with top paper edge positioned about 0.5″-1.5″ above top edge of the round hole, so that all material passing through the round hole from the spray dispenser would be caught by the paper trap. The bottom corners of the paper are weighed down by another pair of binder clips, so that the sprayed liquid accumulated on the plastic sheet from the sprayer side would not drip through the hole onto the paper trap. The nozzle of the spray dispenser is covered by a separate towel and primed two times (by completely squeezing the trigger). The covering towel is then removed.

Required number of full sprays (completely squeezing the trigger and holding the trigger in the squeezed position until the spray stops) is performed, while observing and confirming that the center of each spray cone is passing through the round hole. If the center of a specific spray cone does not pass through the round hole, adjustment is made to the position of the spray dispenser relative to the plastic sheet as described above, and the sprays are repeated by using a new paper trap.

Paper traps are weighed before the sprays using a scale balance with a 1 microgram (6-digit) to 0.1 milligram (4-digit) precision. After the sprays, the paper traps are hung on a string and dried overnight at RH 30-50% and room temperature 65-75 degrees Fahrenheit. The dried paper traps are then weighed by using the same scale balance. The after-drying weights of the paper traps are subtracted with the before-spraying weights thereof, and the resulting measurements from paper traps treated with the same sample packaged fabric refreshening product are averaged or otherwise statistically processed to calculate the total amount of non-volatiles delivered by the sample packaged fabric refreshening product onto the paper trap, which is indicative of the odor control and malodor reduction performance of the sample product. Following is a step-by-step description of the paper trap test method:

First, the paper trap is folded 5 times into about a 1.5″×1.5″ square and pressed so that the last fold would not open when left without pressure to more than 90 degrees to (1) accommodate its placement into the scale balance chamber and (2) enhance its liquid trapping efficiency. Four paper traps for four repetitions per one testing condition (i.e., a sample packaged fabric refreshening product embodying a unique combination of a specific spray dispenser with a specific liquid fabric freshening composition) are prepared. All folded paper traps are uniquely labeled with a fine permanent marker and equilibrated for at least 1 hour in the room air environment in scale balance location with RH 30-50% and room temperature 65-75 degrees Fahrenheit. Each equilibrated paper trap is weighed reading and recording its weight in 4 minutes after its placement into the scale balance chamber and closing its doors.

Second, each paper trap in turn is hung as described in the general description. The nozzle of the spray dispenser is covered by a separate towel and primed two times. The covering towel is then removed, followed by 5 full sprays from the sample packaged fabric refreshening product, while ensuring that the center of the spray cone comes through the round hole.

Third, each spray-treated paper trap is hung on a string and dried overnight (over 14 hours) at RH 30-50% and room temperature 65-75 degrees Fahrenheit.

Fourth, each dried paper trap is folded again 5 times into about a 1.5″×1.5″ square so that their unique labels are visible after the final fold and pressed so that the last fold would not open when left without pressure to more than 90 degrees to accommodate its placement into the scale balance chamber. All folded paper traps are equilibrated for at least 1 hour in the room air environment in scale balance location with RH 30-50% and room temperature 65-75 degrees Fahrenheit. Each equilibrated paper trap is weighed reading and recording its weight in 4 minutes after its placement into the scale balance chamber and closing its doors.

Finally, the difference between weights of the same paper trap measured before the spray treatment and after drying overnight is calculated and recorded as the weight of non-volatiles delivered by the sample packaged fabric refreshening product.

### EXAMPLES

### Example 1: Comparative Examples Showing Improvement in Focused Non-Volatile Delivery by Inventive Packaged Fabric Refreshening Product

One inventive packaged fabric refreshening product (I) and three comparative packaged fabric refreshening products (A-C) are provided, as follows:

The inventive packaged fabric refreshening product (I) comprises the combination of a liquid fabric freshening composition (a) containing a higher level of cyclodextrin (hereinafter “High-CD FR Formula”) with a continuous trigger-activated spray dispenser supplied by Yoshino (hereinafter “Yoshino Sprayer”).

The comparative packaged fabric refreshening product (A) comprises the combination of a liquid fabric freshening composition (b) containing a lower level of cyclodextrin (hereinafter “Low-CD FR Formula”) with a non-continuous trigger-activated spray dispenser supplied by Canyon (hereinafter “Canyon Sprayer”). The comparative packaged fabric refreshening product (B) comprises the combination of the High-CD FR Formula with the Canyon Sprayer. The comparative packaged fabric refreshening product (C) comprises the combination of the Low-CD FR Formula with the Yoshino Sprayer.

Compositional breakdowns of the High-CD and Low-DC FR Formulas are listed below:

TABLE 1

| Ingredients (wt %) | Low-CD FR | High-CD FR |
|---|---|---|
| Ethanol | 3 | 3 |
| Polyethyleneimine (1) | 0.08 | 0.08 |
| Silicone-based wetting agent (2) | 0.12 | 0.12 |
| Didecyldimethyl ammonium chloride (3) | 0.16 | 0.28 |
| Maleic Acid | 0.08 | 0.08 |
| Citric acid | 0.015 | 0.015 |
| Hydroxypropyl beta-cyclodextrin (4) | 0.63 | 1.89 |
| Hydrogenated castor oil (5) | 0.04 | 0.04 |
| Perfume | 0.064 | 0.064 |
| Sodium hydroxide | 0.012 | — |
| Water and minors | To 100% | To 100% |

(1) Lupasol HF from BASF
(2) Silwet L-7600 from Momentive
(3) Bardac 2250J from Lonza
(4) Cavasol W7 HP from Wacker
(5) Basophor EL 60 from BASF

Physical and performance parameters of the Canyon and Yoshino Sprayers are also provided below:

TABLE 2

| | | |
|---|---|---|
| Measurements | Canyon Sprayer | Yoshino Sprayer |

| Lever length mm | 60 | | 60 | |
|---|---|---|---|---|
| Force to Actuate | 35 | | 40 | |
| Strokes to prime | 5 | | 5 | |
| Output per stroke | 1 | ml | 1.1 | ml |
| (at 90 SPM) | | | | |
| Spray duration | <0.2 | seconds | 0.8 | seconds |
| % < 10 μm | 0.23% | | 0.30% | |
| DV10 (μm) | 48.16 | | 43.91 | |
| DV50 (μm) | 102.3 | | 74.95 | |
| DV90 (μm) | 270.2 | | 120.5 | |
| DV[3][2] (μm) | 86.35 | | 64.55 | |
| DV[4][3] (μm) | 183.8 | | 78.79 | |
| FtA 90 SPM N | 35 | | 40 | |
| Spray pattern (at 15 cm) | 18 | cm | 16.5 | cm |

| | | |
|---|---|---|
| Cone angle of mist | 61.9 | 57.6 |

Weights of non-volatiles delivered by such inventive and comparative packaged fabric refreshening products within a focused area are measured by the Paper Trap Method disclosed in Test 3 hereinabove. Each sample packaged fabric refreshening product is repeatedly measured in 4 replicates, and the average results are listed below:

TABLE 3

| | Comparative | | | |
|---|---|---|---|---|
| | Sample (A) | Comparative | Comparative | Inventive |
| | “Control” | Sample (B) | Sample (C) | Sample (I) |
| | Low-CD FR + | High-CD FR + | Low-CD FR + | High-CD FR + |
| | Canyon Sprayer | Canyon Sprayer | Yoshino Sprayer | Yoshino Sprayer |
| Average Weight (g) | 16.8 | 19.4 | 35.5 | 68.9 |
| of Non-Volatiles | | | | |
| Delivered | | | | |
| ΔW | — | 2.6 | 18.7 | 52.1 |
| (Weight Difference | | | | |
| Over Control) | | | | |

It is evident from the above data that the inventive packaged fabric refreshing product (A) employing a combination of High-CD FR Formula with the Yoshino Sprayer surprisingly and unexpectedly results in a synergistic improvement of focused delivery of non-volatiles (which includes cyclodextrin as the odor-absorbing and malodor-control active). Therefore, the inventive packaged fabric refreshing product of the present disclosure provides more effective and efficient deodorization and refreshening of fabric-covered surfaces.

### Example 2: Exemplary High-Cyclodextrin Liquid Fabric Freshening Formulations for Inventive Packaged Fabric Refreshening Products

Following are various exemplary High-CD formulations (1)-(7) that can be used to make the inventive packaged fabric refreshening products of the present disclosure:

TABLE 4

| Ingredients (wt %) | I | 2 | 3 | 4 | 5 | 6 | 7 |
|---|---|---|---|---|---|---|---|
| Ethanol | 3 | 5 | 5 | 5 | 2 | 2 | 1-10 |
| Glycol phenyl ether (1) | — | — | — | — | 0.5 | 1.5 | 0-2 |
| Polyethyleneimine (2) | 0.08 | 0.08 | — | 0.08 | 0.065 | 0.045 | 0-0.1 |
| Silicone-based wetting agent (3) | 0.12 | 0.1 | 0.12 | 0.12 | 0.05 | 0.1 | 0-0.2 |
| Didecyldimethyl ammonium | 0.28 | 0.12 | 0.12 | 0.33 | 0.06 | 0.06 | 0-0.5 |
| chloride (4) | | | | | | | |
| Dioctyldimethyl ammonium | — | 0.12 | 0.12 | — | — | — | 0-0.5 |
| chloride (5) | | | | | | | |
| Maleic Acid | 0.08 | 0.1 | — | 0.08 | 0.06 | 0.06 | 0-0.1 |
| Citric acid | 0.015 | 0.015 | 0.15 | 0.03 | 0.03 | 0.015 | 0.01-0.2 |
| Sodium Citrate | — | — | 0.3 | — | — | — | 0-0.5 |
| Preservative (6) | — | — | — | — | 0.0044 | 0.0044 | 0-0.01 |
| Hydroxypropyl beta- | 1.89 | 1.0 | 2.0 | 2.5 | 1.26 | 1.26 | 1-5 |
| cyclodextrin (7) | | | | | | | |
| Hydrogenated castor oil (8) | 0.04 | 0.3 | 0.3 | 0.2 | 2.5 | 0.4 | 0-5 |
| Perfume | 0.064 | 0.1 | 0.15 | 0.2 | 0.85 | 0.7 | 0-1.5 |
| Sodium hydroxide | — | — | — | — | 0.014 | 0.014 | 0-0.5 |
| Water and minors | To | To | To | To | To | To | Balance |
| | 100% | 100% | 100% | 100% | 100% | 100% | |

(1) Dowanol Eph6 from Dow
(2) Lupasol HF from BASF
(3) Silwet L-7600 from Momentive
(4) Bardac 2250 or Uniquat 2250, from Lonza
(5) Bardac LFE from Lonza
(6) Koralone B-119 from Dupont
(7) Cavasol W7 HP from Wacker
(8) Basophor EL 60 from BASF

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any example disclosed or claimed herein or that it alone, or in any combination with any other reference or references, teaches, suggests or discloses any such example. Further, to the extent that any meaning or definition of a term in this document conflicts with any meaning or definition of the same term in a document incorporated by reference, the meaning or definition assigned to that term in this document shall govern.

While particular examples of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this disclosure.

What is claimed is:

1. A packaged fabric refreshening product comprising:
a liquid fabric freshening composition; and
a spray dispenser for dispensing the liquid fabric freshening composition, wherein:
the liquid fabric freshening composition comprises a liquid carrier and from 1 wt % to 5 wt % of cyclodextrin; and
the spray dispenser is a manually activated trigger-type spray dispenser, which, upon activation by a single movement of a trigger mechanism, is capable of continuously dispensing the liquid fabric freshening composition for a duration of at least 0.4 seconds.

2. The packaged product according to claim 1, wherein the cyclodextrin is selected from the group consisting of alpha-cyclodextrin, beta-cyclodextrin, gamma-cyclodextrin, and derivatives and/or mixtures thereof; wherein optionally the cyclodextrin is selected from the group consisting of hydroxy-propyl alpha-cyclodextrin (HPACD), methylated alpha-cyclodextrin (MACD), methylated beta-cyclodextrin (MBCD), hydroxy-ethyl beta-cyclodextrin (MEBCD), hydroxy-propyl beta-cyclodextrin (HPBCD), and derivatives and/or mixtures thereof.

3. The packaged product according to claim 2, wherein the liquid fabric freshening composition comprises from 1.2 wt % to 3 wt % of cyclodextrin.

4. The packaged product according to claim 2, wherein the liquid fabric freshening composition comprises from 1.5 wt % to 2.5 wt % of cyclodextrin.

5. The packaged product according to claim 1, wherein the liquid fabric freshening composition further comprises one or more ingredients selected from the group consisting of: one or more perfumes; one or more cyclodextrin-compatible surfactants; and one or more cyclodextrin-compatible antimicrobial actives.

6. The packaged product according to claim 1, wherein the spray dispenser is capable of continuously dispensing the liquid fabric freshening composition for a duration of from 0.4 seconds to 2 seconds upon activation by a single movement of the trigger mechanism.

7. The packaged product according to claim 1, wherein the spray dispenser is capable of continuously dispensing the liquid fabric freshening composition for a duration of from 0.5 seconds to 1.8 seconds upon activation by a single movement of the trigger mechanism.

8. The packaged product according to claim 1, wherein the spray dispenser is capable of continuously dispensing the liquid fabric freshening composition for a duration of from 0.6 seconds to 1.5 seconds upon activation by a single movement of the trigger mechanism.

9. The packaged product according to claim 1, wherein the spray dispenser comprises:
a container body in which the liquid fabric freshening composition is contained;
a nozzle; and
an ejector in fluid communication with the container body and the nozzle for suctioning the liquid fabric freshening composition from the container body and for ejecting the liquid fabric freshening composition forward through a nozzle upon activation by the trigger mechanism.

10. The packaged product according to claim 1, wherein the dispensed liquid fabric freshening composition is characterized by an average droplet size (DV50) of from 50 microns to 120 microns.

11. The packaged product according to claim 1, wherein the dispensed liquid fabric freshening composition is characterized by an average droplet size (DV50) of from 60 microns to 110 microns.

12. The packaged product according to claim 1, wherein the dispensed liquid fabric freshening composition is characterized by an average droplet size (DV50) of from 70 microns to 100 microns.

13. A method of refreshening a fabric surface and reducing malodor therefrom, comprising the steps of:
providing a liquid fabric freshening composition that comprises a liquid carrier and from 1 wt % to 5 wt % of cyclodextrin; and
spraying an effective amount of the liquid fabric freshening composition onto a fabric surface by using a manually activated trigger-type spray dispenser, wherein:
the fabric surface is substantially planar with a minimal dimension of no less than 0.6 meters; and
the manually activated trigger-type spray dispenser, upon activation by a single movement of a trigger mechanism, is capable of continuously dispensing the liquid fabric freshening composition for a duration of at least 0.4 seconds.

14. The method of claim 13, wherein the fabric surface is selected from the group consisting of beddings, mattresses, sofas, and carpets.
